(19) United States
(12) Patent Application Publication
(10) Pub. No.: US 2012/0271056 A1
(43) Pub. Date: Oct. 25, 2012

(54) PROCESS FOR THE PREPARATION OF CRYSTALLINE FORM I OF L-MALIC ACID SALT OF SUNITINIB

(75) Inventors: Sudhir Singh Sanwal, Kangra (IN); Saridi Madhava Dileep Kumar, Vizianagaram (IN); Swargam Sathyanarayana, Karim Nagar (IN); Rajesh Kumar Thaper, Jammu (IN); Mohan Prasad, Gurgaon (IN)

(73) Assignee: RANBAXY LABORATORIES LIMITED, New Delhi, Delhi (IN)

(21) Appl. No.: 13/508,907

(22) PCT Filed: Nov. 12, 2010
(86) PCT No.: PCT/IB10/55150

§ 371 Date: Jun. 22, 2012

(30) Foreign Application Priority Data
Nov. 12, 2009 (IN) 2337/DEL/2009

(51) Int. Cl. C07D 209/46; A61P 1/00; A61K 31/4035

(52) U.S. Cl. 548/465

(57) ABSTRACT

The present invention relates to a process for the preparation of crystalline Form I of the L-malic acid salt of sunitinib.

### FIELD OF THE INVENTION

The present invention relates to a process for the preparation of crystalline Form I of the L-malic acid salt of sunitinib.

### BACKGROUND OF THE INVENTION

Sunitinib is chemically described as N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide as represented by Formula I.

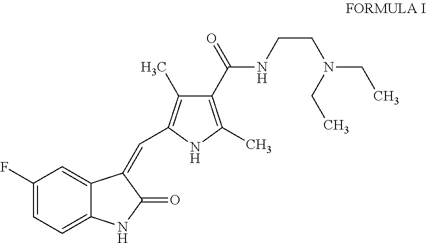

Sunitinib is an oral multi-kinase inhibitor and useful for the treatment of gastrointestinal stromal tumor and advanced renal cell carcinoma. Sunitinib is commercially available as the L-malate salt, which is described chemically as butanedioic acid, hydroxy-, (2S)-, compound with N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide (1:1).

U.S. Publication Nos. 2003/0069298 and 2007/0191458 describe the preparation of crystal Form I and Form II of L-malic acid salt of sunitinib. According to these publications, crystal Form I of L-malic acid salt of sunitinib is prepared by slurrying a poorly crystalline or crystal Form II of L-malic acid salt of sunitinib in acetonitrile. Acetonitrile, methanol, ethanol, isopropanol, toluene, n-butanol, tetrahydrofuran, N,N-dimethylformamide, acetone and water are mentioned as useful solvents for preparing the crystal Form I in U.S. Publication No. 2003/0069298. Crystalline Form II of the L-malic acid salt of sunitinib is prepared by dissolving the crystalline Form I of the L-malic acid salt of sunitinib in tetrahydrofuran and water and allowing the solvent to evaporate overnight.

WO 2009/067686 describes processes for preparing crystalline forms of racemic sunitinib malate, sunitinib hemi-L-malate and compositions containing sunitinib base and L- or racemic malic acid.

WO 2009/104021 describes processes for preparing crystalline Form III and Form IV of sunitinib L-malate. WO 2009/104021 discloses that Form II of sunitinib L-malate is hygroscopic, thermodynamically unstable and appears to readily convert to Form I.

### SUMMARY OF THE INVENTION

In one general aspect, the present invention provides for a process for the preparation of crystalline Form I of the L-malic acid salt of sunitinib. The process includes:
a) contacting L-malic acid with sunitinib base in a solvent; and
b) isolating crystalline Form I of the L-malic acid salt of sunitinib from the mixture thereof.

Embodiments of this aspect may include one or more of the following features. For example, the solvent may be water, an organic solvent, or a mixture thereof. Suitable organic solvents include alkanols, esters, nitriles, aromatic hydrocarbons, cyclic ethers, or ketones, or a mixture thereof. Suitable alkanols include n-propanol, methanol, ethanol, isopropanol or n-butanol. Suitable esters include n-butyl acetate, isopropyl acetate, methyl acetate or ethyl acetate.

The L-malic acid is contacted with sunitinib base in solvent at temperature of about 15° C. to 80° C. or the L-malic acid is contacted with sunitinib base in solvent at temperature of about 55° C. to 70° C.

In another general aspect, the present invention provides for a process for the preparation of crystalline Form I of the L-malic acid salt of sunitinib. The process includes crystallizing L-malic acid salt of sunitinib from a solvent selected from the group consisting of n-propanol, n-butyl acetate, isopropyl acetate, methyl acetate and ethyl acetate.

### BRIEF DESCRIPTION OF THE DRAWINGS

FIG. 1 depicts the X-ray powder diffraction pattern (XRPD) of crystalline Form I of the L-malic acid salt of sunitinib.

FIG. 1A provides a table of values for the XRPD pattern depicted in FIG. 1.

### DETAILED DESCRIPTION OF THE INVENTION

The term “L-malic acid salt of sunitinib” includes a combination of sunitinib and L-malic acid in any molar ratio between about 1:0.75 and about 1:1.5.

The present invention provides for a process for the preparation of crystalline Form I of L-malic acid salt of sunitinib. The process includes:
a) contacting L-malic acid with sunitinib base in a solvent; and
b) isolating crystalline Form I of the L-malic acid salt of sunitinib from the mixture thereof.

The sunitinib base may be prepared according to the method provided in U.S. Pat. No. 6,573,293. L-Malic acid is contacted with sunitinib base in a solvent. The solvent may be water or an organic solvent, or a mixture thereof. The organic solvent may be an alkanol, for example, n-propanol, methanol, ethanol, isopropanol or n-butanol; an ester, for example, n-butyl acetate, isopropyl acetate, methyl acetate or ethyl acetate; a nitrile, for example, acetonitrile; an aromatic hydrocarbon, for example, toluene; a cyclic ether, for example, tetrahydrofuran; or a ketone, for example, acetone, or a mixture thereof.

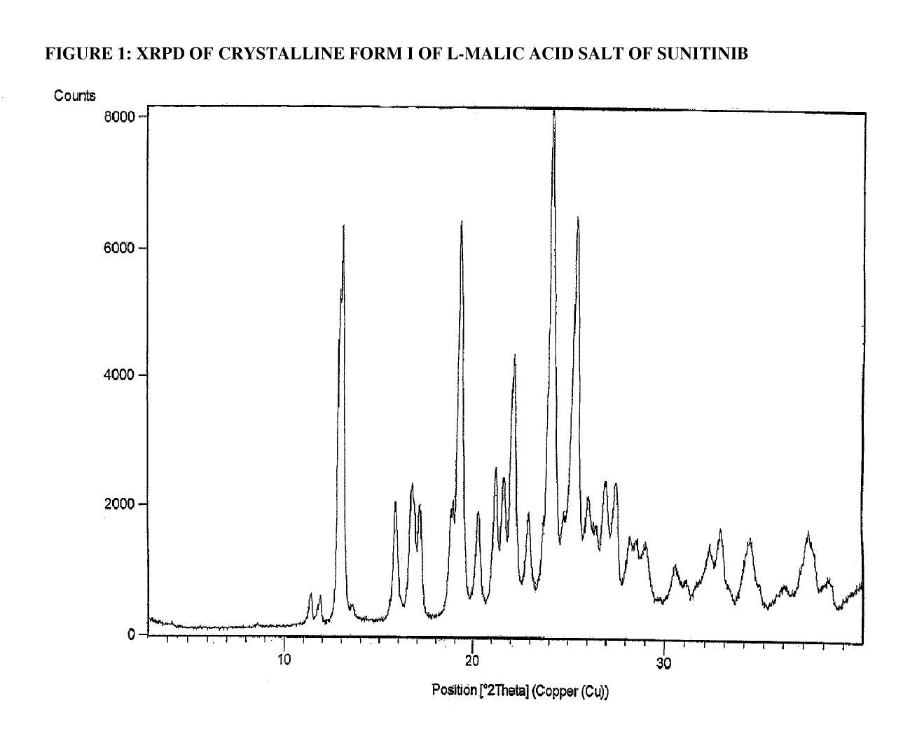

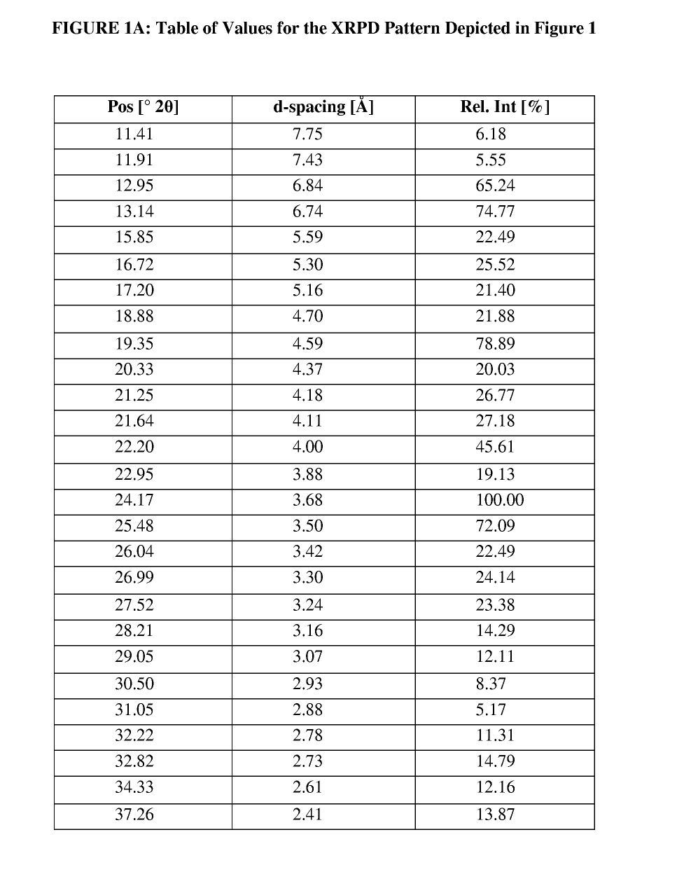

The L-Malic acid is contacted with sunitinib base in a solvent at a temperature of about 15° C. to about 80° C. For example, the L-Malic acid is contacted with sunitinib base in a solvent at a temperature of about 55° C. to about 70° C., followed by a temperature of about 15° C. to about 30° C. The formation of crystalline Form I of the L-malic acid salt of sunitinib may be facilitated by stirring the reaction mixture for a sufficient time, for example, for about 1 hour to about 50 hours. Crystalline Form I of the L-malic acid salt of sunitinib may be isolated by filtration, decantation, evaporation, distillation, or a combination thereof. Crystalline Form I of the L-malic acid salt of sunitinib has substantially the same XRPD pattern as depicted in FIG. 1.

The present invention also provides for a process for the preparation of crystalline Form I of the L-malic acid salt of sunitinib. The process includes crystallizing L-malic acid salt of sunitinib from a solvent selected from the group consisting of n-propanol, n-butyl acetate, isopropyl acetate, methyl acetate and ethyl acetate.

XRPD of the samples were determined by using a Panalytical X'Pert Pro X-Ray Powder Diffractometer in the range 3-40 degree 2 theta and under tube voltage and current of 45 Kv and 40 mA, respectively. Copper radiation of wavelength 1.54 angstrom and Xceletor detector was used.

While the present invention has been described in terms of its specific embodiments, certain modifications and equivalents will be apparent to those skilled in the art and are intended to be included within the scope of the present invention.

### EXAMPLES

### Example 1

### Preparation of Crystalline Form I of the L-Malic Acid Salt of Sunitinib

Sunitinib base (2 g) was added to n-propanol (50 ml) and stirred for 30 minutes. L-Malic acid (0.7 g) was added to the mixture and stirred at 60° C. to 65° C. for 2 hours. The mixture was further stirred at about 20° C. to 25° C. for 18 hours, filtered under vacuum at 100 mBar to 150 mBar and dried under vacuum at 800 mBar to 900 mBar at 55° C. to 60° C. to obtain the title compound.

Yield: 2.6 g

Moisture content: 0.25%

### Example 2

### Preparation of Crystalline Form I of the L-Malic Acid Salt of Sunitinib

Sunitinib base (2 g) was added to n-butyl acetate (50 ml) and stirred for 30 minutes. L-Malic acid (0.7 g) was added to the mixture and stirred at 60° C. to 65° C. for 2 hours. The mixture was further stirred at about 20° C. to 25° C. for 18 hours, filtered under vacuum at 100 mBar to 150 mBar and dried under vacuum at 800 mBar to 900 mBar at 55° C. to 60° C. to obtain the title compound.

Yield: 2.5 g

Moisture content: 0.45%

### Example 3

### Preparation of Crystalline Form I of the L-Malic Acid Salt of Sunitinib

Sunitinib base (2 g) was added to isopropyl acetate (50 ml) and stirred for 30 minutes. L-Malic acid (0.7 g) was added to the mixture and stirred at 60° C. to 65° C. for 2 hours. The mixture was further stirred at about 20° C. to 25° C. for 18 hours, filtered under vacuum at 100 mBar to 150 mBar and dried under vacuum at 800 mBar to 900 mBar at 50° C. to 55° C. to obtain the title compound.

Yield: 2.6 g

Moisture content: 0.38%

### Example 4

### Preparation of Crystalline Form I of the L-Malic Acid Salt of Sunitinib

Sunitinib base (2 g) was added to methyl acetate (50 ml) and stirred for 30 minutes. L-Malic acid (0.7 g) was added to the mixture and stirred at 60° C. to 65° C. for 2 hours. The mixture was further stirred at about 20° C. to 25° C. for 18 hours, filtered under vacuum at 100 mBar to 150 mBar and dried under vacuum at 800 mBar to 900 mBar at 50° C. to obtain the title compound.

Yield: 2.4 g

Moisture content: 0.48%

### Example 5

### Preparation of Crystalline Form I of the L-Malic Acid Salt of Sunitinib

Sunitinib base (2 g) was added to ethyl acetate (50 ml) and stirred for 30 minutes. L-Malic acid (0.7 g) was added to the mixture and stirred at 60° C. to 65° C. for 2 hours. The mixture was further stirred at about 20° C. to 25° C. for 18 hours, filtered under vacuum at 100 mBar to 150 mBar and dried under vacuum at 800 mBar to 900 mBar at 50° C. to obtain the title compound.

Yield: 2.5 g

Moisture content: 0.47%

We claim:

1. A process for the preparation of crystalline Form I of the L-malic acid salt of sunitinib, wherein the process comprises:
a) contacting L-malic acid with sunitinib base in a solvent; and
b) isolating crystalline Form I of the L-malic acid salt of sunitinib from the mixture thereof.

2. A process according to claim 1, wherein the solvent comprises water, an organic solvent, or a mixture thereof.

3. A process according to claim 2, wherein the organic solvent comprises an alkanol, an ester, a nitrile, an aromatic hydrocarbon, a cyclic ether, or a ketone, or a mixture thereof.

4. A process according to claim 3, wherein the alkanol comprises n-propanol, methanol, ethanol, isopropanol or n-butanol.

5. A process according to claim 3, wherein the ester comprises n-butyl acetate, isopropyl acetate, methyl acetate or ethyl acetate.

6. A process according to claim 1, wherein the L-malic acid is contacted with sunitinib base in solvent at temperature of about 15° C. to 80° C.

7. A process according to claim 6, wherein the L-malic acid is contacted with sunitinib base in solvent at temperature of about 55° C. to 70° C.

8. A process for the preparation of crystalline Form I of the L-malic acid salt of sunitinib, wherein the process comprises crystallizing L-malic acid salt of sunitinib from a solvent selected from the group consisting of n-propanol, n-butyl acetate, isopropyl acetate, methyl acetate and ethyl acetate.
